(19) United States
(12) Patent Application Publication
(10) Pub. No.: US 2025/0051787 A1
(43) Pub. Date: Feb. 13, 2025

(54) METHOD FOR GENE EXPRESSION BY TRANSIENT TRANSFORMATION OF WILD RICE SEED USING AGROBACTERIUM

(71) Applicants: Tobacco Research Institute of CAAS, Shandong (CN); Institute of Crop Sciences, CAAS, Beijing (CN)

(72) Inventors: Ning YAN, Shandong (CN); Yi SUI, Shandong (CN); Yali LI, Shandong (CN); Chuanyin WU, Shandong (CN); Wanhong LI, Shandong (CN); Qing MA, Shandong (CN); Hongbo ZHANG, Shandong (CN); Zhongfeng ZHANG, Shandong (CN)

(73) Assignees: Tobacco Research Institute of CAAS, Shandong (CN); Institute of Crop Sciences, CAAS, Beijing (CN)

(21) Appl. No.: 18/652,906

(22) Filed: May 2, 2024

(30) Foreign Application Priority Data
Aug. 9, 2023 (CN) 202310994490.0

(51) Int. Cl. C12N 15/82

(52) U.S. Cl. CPC C12N 15/8205; C12N 15/8242

(57) ABSTRACT

Disclosed is a method for gene expression by transient transformation of a wild rice seed using Agrobacterium. The method includes the following steps: subjecting a wild rice seed to induction culture and subculture in sequence, and selecting a resulting callus with a dense structure to allow pre-culture to obtain a pre-cultured callus; transforming a 1305Ubi-Ubi-GFP-H plasmid carrying a green fluorescent protein (GFP) gene into an Agrobacterium strain EHA105 or LBA4404 to obtain a positive Agrobacterium strain; subjecting the positive Agrobacterium strain to culture to obtain a bacterial suspension with an optical density (OD) value of 0.02 to 0.5 at a wavelength of 600 nm to obtain an infection bacterial solution; and infecting the pre-cultured callus with the infection bacterial solution to allow co-culture and recovery culture in sequence; observing an infection status of a recovered callus under a stereo fluorescence microscope, and calculating a transient expression rate.

### CROSS-REFERENCE TO RELATED APPLICATION

This patent application claims the benefit and priority of Chinese Patent Application No. 202310994490.0 filed with the China National Intellectual Property Administration on Aug. 9, 2023, the disclosure of which is incorporated by reference herein in its entirety as part of the present application.

### TECHNICAL FIELD

The present disclosure belongs to the technical field of genetic engineering, and specifically relates to a method for gene expression by transient transformation of a wild rice seed using Agrobacterium.

### BACKGROUND

Wild rice seed as the caryopsis of Zizania latifolia (Griseb.) Turcz. ex Stapf is a specialty crop with a long history. The wild rice seed is an important cereal with the homology of medicine and food. In the Li Shizhen's “Compendium of Materia Medica” in Ming Dynasty and other works, there are records of assistance in the treatment of diabetes mellitus and gastrointestinal diseases using the wild rice seed. In recent years, the research on nutritional components of Zizania has mainly focused on its seeds, namely the wild rice seeds. Wild rice seed is not only rich in nutrients such as proteins, amino acids, vitamins, and minerals, but also contains biologically-active substances such as resistant starch, dietary fiber, phytosterols, anthocyanins, and proanthocyanidins. Compared with rice, the wild rice seed is a high-protein and low-fat health food, and shows a biological activity and health care effects that have been widely concerned by East Asian scientists from China and South Korea. The proven biological activities and health care values of wild rice seed include antioxidant activity, improvement of insulin resistance and lipotoxicity, as well as prevention of cardiovascular diseases. However, Zizania latifolia also demonstrates some inherent limitations such as easy shattering of wild rice seed, excessive plant height, unfocused flowering period, and low seed setting rate, making it unsuitable for large-scale production applications.

Transient expression refers to a technology that introduces a target gene into recipient cells to establish a temporary high-efficiency expression system, thereby allowing large amounts of target genes to be expressed in a relatively short period of time. Transient expression does not require the integration of exogenous genes into the chromosomes of cells, does not require selection, and does not produce offspring that can be stably inherited. Therefore, compared with stable expression, the transient expression shows simplicity, rapidity, short cycle, high efficiency, and strong biological safety. The inventors and their team take advantages of the transient expression to establish a transient transformation system for the wild rice seed. This system can not only improve transformation efficiency of the wild rice seed, but also lay a technical foundation for better subsequent research on functional genes in the wild rice seed.

The inventor's team established a method for plant regeneration from wild rice callus (first Chinese patent application CN112753580A). The explants (wild rice seeds) used are easy to obtain, and the preserved wild rice seeds can be used at any time without seasonal restrictions. By using a modified basic medium and optimized culture conditions, robust regenerated plants can be obtained in just 3 months. Subsequently, during the research on functional genes of wild rice, a method for establishing a genetic transformation system of wild rice seed was proposed (the second Chinese patent application CN113637701A). Based on the first Chinese patent application of wild rice, a regeneration efficiency of wild rice callus has been greatly improved by optimizing the culture conditions and culture methods of wild rice embryogenic callus and optimizing the medium for callus regeneration. This process provides high-quality embryogenic callus receptor materials for Agrobacterium infection and then lays a solid foundation for transformation. In addition, the treatment methods and culture conditions during the transformation are explored and optimized through experiments to obtain transformed positive strains, thus successfully establishing a stable Agrobacterium-mediated genetic transformation system for the wild rice. However, the method for establishing a genetic transformation system of wild rice seed has low genetic transformation efficiency on the wild rice seed. Based on this, the present disclosure is proposed.

### SUMMARY

To solve the above-mentioned problems existing in the prior art, a purpose of the present disclosure is to propose a method for gene expression by transient transformation of a wild rice seed using Agrobacterium. In the present disclosure, the wild rice seed is subjected to transient transformation with Agrobacterium containing a green fluorescent protein (GFP) plasmid to optimize an appropriate bacterial species, a bacterial concentration, and a co-culture time for infection, and improve efficiency of transient transformation of wild rice, and realize expression and visual observation of an exogenous gene in the wild rice. This method has laid the technical foundation for better subsequent research on functional genes in the wild rice.

The technical solutions of the present disclosure are as follows.

The present disclosure provides a method for gene expression by transient transformation of a wild rice seed using Agrobacterium, including the following steps:
(1) inoculating a sterilized wild rice seed into an induction medium to allow induction culture of a callus, conducting subculture, and selecting a resulting callus with a dense structure to allow pre-culture to obtain a pre-cultured callus;
(2) transforming a 1305Ubi-Ubi-GFP-H plasmid carrying a green fluorescent protein (GFP) gene into an Agrobacterium strain EHA105 or LBA4404 to allow culture at 28° C. for 2 d, selecting a resulting single clone to allow PCR and electrophoresis detection to obtain a positive Agrobacterium strain; transferring the positive Agrobacterium strain into a yeast extract broth (YEB) liquid medium to allow culture, subjecting an obtained bacterial solution to centrifugation to collect a bacterial cell, resuspending the bacterial cell in an infection buffer until a resulting bacterial suspension has an optical density (OD) value of 0.02 to 0.5 at a wavelength of 600 nm to obtain an infection bacterial solution; and
(3) infecting the pre-cultured callus with the infection bacterial solution, inoculating a resulting infected callus into a co-culture medium to allow co-culture in the dark at 22° C.±2° C. for 2 d to 4 d; transferring a resulting co-cultured callus into a recovery medium to allow recovery culture in the dark at 28° C.±2° C. for 3 d; observing an infection status of a recovered callus under a stereo fluorescence microscope (Leica M305FCA fluorescence stereo microscope equipped with a DMC6200 camera), where the GFP gene serving as an exogenous target gene is successfully expressed in the callus of the wild rice seed if the recovered callus appears green fluorescence under ultraviolet light; and calculating a transient expression rate.

The transient expression rate of the GFP gene has a calculation formula as follows: GFP transient expression rate (%)=number of GFP-positive callus grains/number of infected callus grains×100%.

The infected callus is inoculated into the co-culture medium to allow the co-culture for 2 d to 4 d, such as 2 d, 2.5 d, 3 d, 3.5 d, or 4 d. However, the co-culture time is not limited to the listed values, and other unlisted values within this range are also applicable.

The bacterial suspension has an OD value of 0.02 to 0.5 at a wavelength of 600 nm, such as 0.02, 0.05, 0.1, 0.15, 0.18, 0.2, 0.22, 0.25, 0.3, 0.35, 0.4, 0.45, and 0.5. However, the OD value is not limited to the listed values, and other unlisted values within this range are also applicable.

Further, the Agrobacterium strain is any one selected from the group consisting of Agrobacterium strains EHA105 and LBA4404, the co-culture is conducted for 3 d, and the bacterial suspension has an OD value of 0.2 at a wavelength of 600 nm.

Further, the Agrobacterium strain is any one selected from the group consisting of Agrobacterium strains EHA105 and LBA4404, the co-culture is conducted for 2 d, and the bacterial suspension has an OD value of 0.2 at a wavelength of 600 nm.

Further, a preparation process of the sterilized wild rice seed in step (1) includes sterilization of explants: immersing a suitable wild rice seed in 75% ethanol for 30 s to 60 s and in 20% sodium hypochlorite for 20 min in sequence, washing a resulting immersed wild rice seed with sterile water 5 to 7 times until an obtained washing solution is clear, and then immersing a resulting washed wild rice seed in the sterile water overnight.

The induction culture includes: an embryo of the sterilized wild rice seed is cut under a microscope with a scalpel, and then inoculated into the induction medium to allow germination, and the induction culture of the callus is conducted at 28° C.±2° C. for 4 to 5 weeks in the dark to obtain an embryogenic callus.

The subculture includes: sprouts and vitrified callus are removed from resulting young embryos, an embryogenic callus with a light yellow appearance and a dense structure is selected to allow the subculture on the induction medium in the dark at 28° C.±2° C.; a resulting subcultured embryogenic callus with a dense structure is selected to allow the pre-culture in the dark at 28° C.±2° C. for 7 d to 9 d.

Further, the induction medium in step (1) includes: 4.43 g of a Murashige & Skoog basal salt mixture (MS salt), 1 mL of 2 mg/mL 2,4-dichlorophenoxyacetic acid (2,4-D), 0.5 g of casein hydrolysate, 30 g of sucrose, 0.1 g of inositol, and 3 g of phytagel per liter of the induction medium with a pH value of 5.7.

In step (2), the plasmid carrying the exogenous target gene is transformed into Agrobacterium competent cells, where the Agrobacterium competent cells are EHA105 Agrobacterium strain, and the plasmid is the 1305Ubi-Ubi-GFP-H plant expression vector containing GFP; the cells are cultured in a constant-temperature incubator at 28° C., and single clones are selected; and a positive Agrobacterium strain is obtained.

Further, 50 mg/mL kanamycin and 50 mg/mL rifampicin are added into the YEB liquid medium in step (2). The positive Agrobacterium strain single clone is transferred into a YEB liquid medium and cultured in a shaker overnight. A resulting shaken bacterial solution is centrifuged under a room temperature at 1,000 rpm to 2,000 rpm for 5 min. After the centrifugation is completed, a resulting supernatant is discarded and obtained collected bacterial cells are resuspended in an infection buffer.

Further, each liter of the infection buffer in step (2) includes 4 g of trace element-containing N6, 1 mL of 200 μM/mL acetosyringone, 200 μL of 10 mg/mL 2,4-D, 1 g of casein hydrolysate, 30 g of sucrose, 0.1 g of inositol, and 10 g of glucose.

Further, the pre-cultured callus is infected with the infection bacterial solution for 20 min, and the infected callus is co-cultured on the co-culture medium for 3 d in step (3).

Further, the co-culture medium in step (3) includes: 4 g of trace element-containing N6, 1 mL of 200 μM/mL acetosyringone, 200 μL of 10 mg/mL 2,4-D, 1 g of casein hydrolysate, 30 g of sucrose, 0.1 g of inositol, 10 g of glucose, and 3 g of phytagel per liter of the co-culture medium with a pH value of 5.7.

Further, the recovery medium in step (3) includes: 4.43 g of MS salt, 0.5 g of casein hydrolysate, 2 mL of 2 mg/mL 2,4-D, 100 μL of 1 mg/mL 6-BA, 2 mL of 200 mg/mL Timentin, 0.1 g of inositol, 30 g of sucrose, and 3 g of phytagel per liter of the recovery medium with a pH value of 5.7.

In step (3), callus recovery culture and GFP observation are conducted after the co-culture is completed: transferring a resulting co-cultured callus evenly and sparsely into a recovery medium to allow recovery culture in the dark at 28° C.±2° C. for 3 d; observing an infection status of a recovered callus under a stereo fluorescence microscope.

The present disclosure has following beneficial effects:

In the present disclosure, the wild rice seed is subjected to transient transformation with Agrobacterium containing a GFP plasmid to optimize an appropriate bacterial species, a bacterial concentration, and a co-culture time for infection, and improve a transient transformation efficiency of wild rice, and realize gene expression and visual observation of an exogenous gene in the wild rice. This method has laid technical foundation for better subsequent research on functional genes in the wild rice.

The wild rice callus transferred into 1305Ubi-Ubi-GFP-H can emit green fluorescence under the stimulation of ultraviolet light. Accordingly, a stereo fluorescence microscope is used to observe the green fluorescence of transient transformation-derived callus of the wild rice seed, thereby identifying the successful expression of the exogenous target gene in the wild rice callus. The identification method is simple, fast, and efficient.

### BRIEF DESCRIPTION OF THE DRAWINGS

FIG. 1A-FIG. 1D show a process of callus induction, where FIG. 1A is a sample appearance of the selected wild rice seed; FIG. 1B shows the step of inoculating the sterilized wild rice seed on an induction medium to allow callus induction; FIG. 1C is a primary callus of the wild rice seed; and FIG. 1D is an embryogenic callus of the wild rice seed.

FIG. 2 shows a physical map of the 1305Ubi-Ubi-GFP-H plasmid vector.

FIG. 3 shows comparison and analysis of a transient expression rate of the EHA105-positive Agrobacterium bacterial solution in callus under different OD values (0.02, 0.05, 0.1, 0.2, and 0.5) at a wavelength of 600 nm and different co-culture times (2 d, 3 d, and 4 d); and

FIG. 4A-FIG. 4F show images of the transient expression of callus fluorescence observed under a stereo fluorescence microscope on days 2, 3, and 4 of co-culture, where FIG. 4A shows a fluorescence expression level of callus on day 2 of co-culture under ultraviolet light; FIG. 4B is a state of the callus on day 2 of co-culture under white light; FIG. 4C is a fluorescence expression level of callus on day 3 of co-culture under ultraviolet light; FIG. 4D is a state of the callus on day 3 of co-culture under white light; FIG. 4E is a fluorescence expression level of callus on day 4 of co-culture under ultraviolet light; and FIG. 4F is a state of the callus on day 4 of co-culture under white light.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

In order to further understand the present disclosure, the present disclosure will be further described in conjunction with the accompanying drawings and examples.

The equipment and reagents used in the examples are all commercially available, unless otherwise specified.

The biological material wild rice seed used in the examples is collected from Baimahu Village, Jinhu County, Huaian City, Jiangsu Province.

### Example 1

This example provided a method for gene expression by transient transformation of a wild rice seed using Agrobacterium, including the following steps:

### Sterilization of Explants:

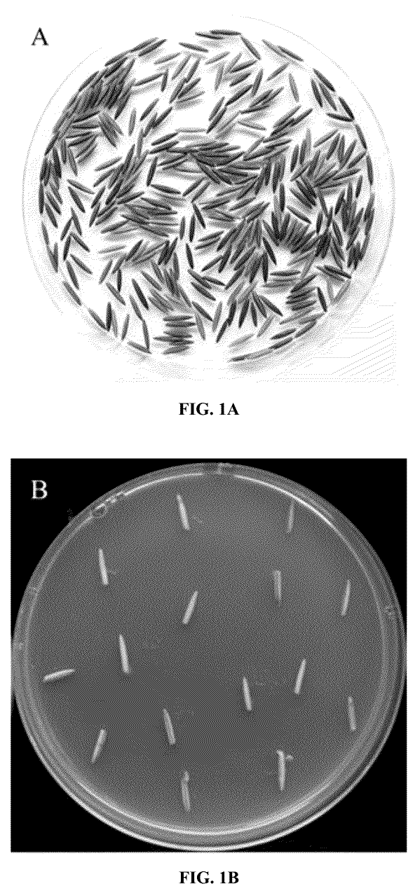

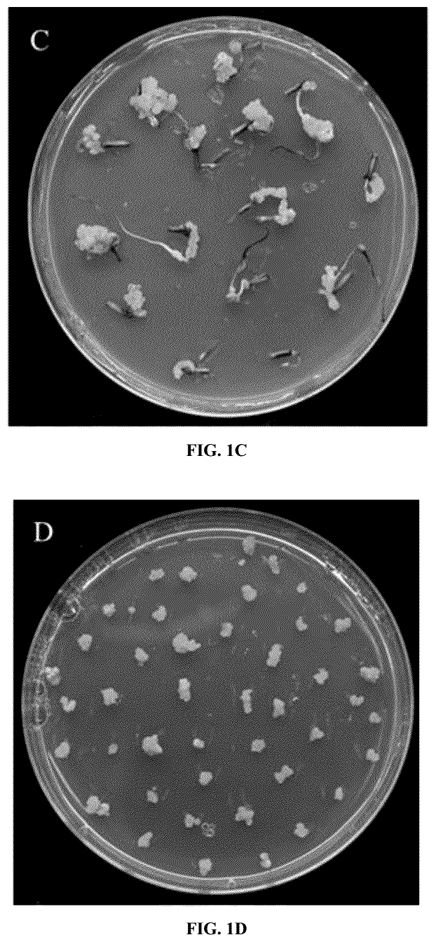

The husks of wild rice seeds were removed, and seeds that were clean and plump, consistent in size, smooth on the surface, and free of mold and spots were selected (FIG. 1A). The seeds were cleaned on the surface with 75% alcohol and then immersed in 75% ethanol for 30 s to 60 s, rinsed 2 times with sterile water and a waste liquid was discarded. The seeds were immersed in 20% sodium hypochlorite for 20 min, during which the seeds were placed in a rotary oscillator at 130 rpm to fully contact the seeds with the disinfectant. The waste liquid after immersing was discarded, and the seeds were washed with sterile water 5 to 7 times, and then sterilized water was added to allow shaking in the rotary oscillator at 130 rpm for 3 h. The seeds were washed with sterile water 3 to 5 times until a washing solution was clear, and then immersed in the sterile water overnight.

### Induction Culture:

An embryo of the sterilized wild rice seed was cut under a microscope with a scalpel, and then inoculated into an induction medium MS containing 2 mg/L 2,4-D and at pH=5.7 (FIG. 1B), and the induction culture of the callus was conducted at 28° C.±2° C. for 4 to 5 weeks in the dark to obtain an embryogenic callus (FIG. 1C).

### Subculture:

The subculture included: sprouts and vitrified callus were removed from resulting young embryos, an embryogenic callus with a light yellow appearance and a dense structure was selected to allow the subculture on the induction medium in the dark at 28° C.±2° C. A resulting subcultured embryogenic callus with a dense structure was selected to allow pre-culture in the dark for 7 d to 9 d before the Agrobacterium co-culture (FIG. 1D).

### Obtaining a Positive Agrobacterium Strain:

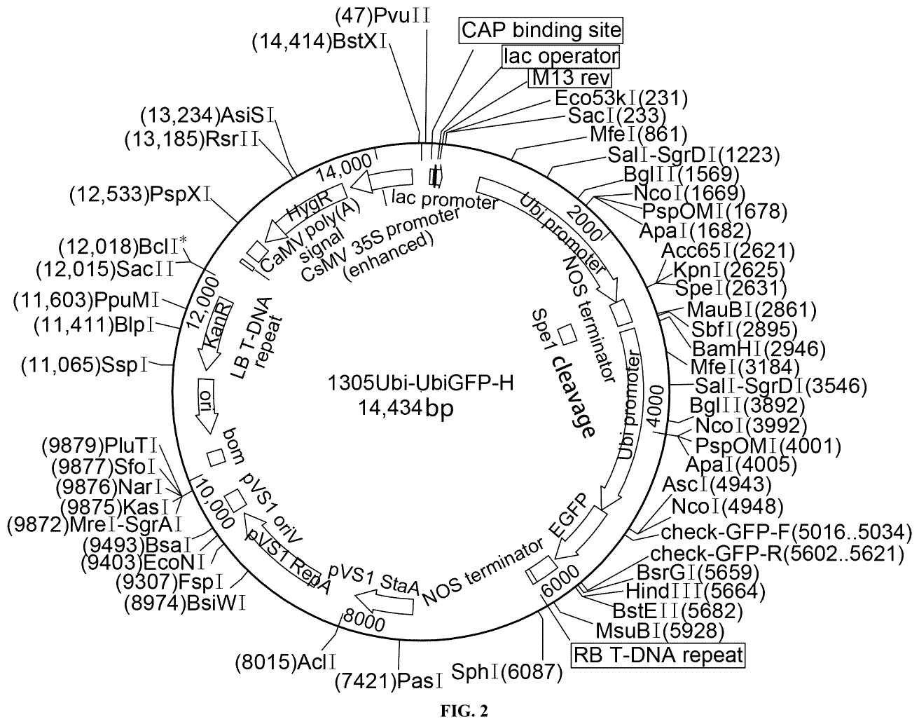

A 1305Ubi-Ubi-GFP-H plasmid (FIG. 2) was transformed into EHA105 Agrobacterium strain, and then single clones were selected to allow PCR and electrophoresis detection to obtain the positive Agrobacterium strain.

### Preparation of a Positive Agrobacterium Bacteria Solution:

The positive single clone colonies were transferred into a YEB liquid medium (including 1 g/L yeast extract, 5 g/L peptone, 5 g/L beef extract, and 0.493 g/L MgSO₄·7H₂O, pH=7.0) containing 50 mg/mL kanamycin and 50 mg/mL rifampicin, and then cultured overnight in a shaker (28° C., 200 rpm). A resulting shaken bacterial solution was centrifuged at 1,500 rpm for 5 min at room temperature, a supernatant was discarded, and bacterial cells were collected. The bacterial cells were resuspended using an infection buffer, where each liter of the infection buffer specifically included 4 g of trace element-containing N6, 1 mL of 200 μM/mL acetosyringone, 200 μL of 10 mg/mL 2,4-D, 1 g of casein hydrolysate, 30 g of sucrose, 0.1 g of inositol, and 10 g of glucose. The bacterial solution after resuspension had an OD value of 0.2 at a wavelength of 600 nm to obtain an infection bacterial solution, which was allowed to stand at room temperature for 0.5 h to 1 h.

Infection with the Positive Agrobacterium Bacterial Solution:

The callus in well conditions and vigorously dividing after 7 d to 9 d of pre-culture was collected into a 50 mL sterile centrifuge tube with a small spoon, and then infected with the infection bacterial solution for 20 min under shaking gently.

### Co-Culture of Callus:

The infection bacterial solution was discarded, and a resulting infected callus was poured into a 90 mm×25 mm disposable petri dish added with one piece of filter paper and two pieces of absorbent paper. The residual bacterial solution was drained on the surface of the callus by gently shaking the petri dish, and the small particles of callus were inoculated into the co-culture medium for 3 d of co-culture at 22° C.±2° C. in the dark.

### Recovery Culture and GFP Observation of Callus:

After the co-culture was completed, the co-cultured callus was evenly and sparsely transferred to a recovery medium to allow recovery culture in the dark at 28° C.±2° C. for 3 d; an infection status of a recovered callus was observed under a stereo fluorescence microscope, where the GFP gene serving as an exogenous target gene was successfully expressed in the callus of the wild rice seed if the recovered callus appeared green fluorescence under ultraviolet light; and a transient expression rate was calculated, where the transient expression rate of GFP was 84%.

### Example 2

This example differed from Example 1 in that the co-culture time of bacterial solution and callus was 2 d.

Results: the callus after culture recovery was placed under a stereo fluorescence microscope to observe the infection status. Green fluorescence showed that the exogenous target gene was successfully expressed in the wild rice callus, and the transient expression rate of GFP was calculated to be 83%.

### Example 3

This example differed from Example 1 in that the LBA4404 Agrobacterium strain was used.

Results: the callus after culture recovery was placed under a stereo fluorescence microscope to observe the infection status. Green fluorescence showed that the exogenous target gene was successfully expressed in the wild rice callus, and the transient expression rate of GFP was calculated to be 83%.

### Example 4

This example differed from Example 1 in that the LBA4404 Agrobacterium strain was used, and the co-culture time of bacterial solution and callus was 2 d.

Results: the callus after culture recovery was placed under a stereo fluorescence microscope to observe the infection status. Green fluorescence showed that the exogenous target gene was successfully expressed in the wild rice callus, and the transient expression rate of GFP was calculated to be 82%.

### Example 5

This example differed from Example 1 in that the bacterial solution had an OD value of 0.02 at a wavelength of 600 nm, and the co-culture time of bacterial solution and callus was 4 d.

Results: the callus after culture recovery was placed under a stereo fluorescence microscope to observe the infection status. Green fluorescence showed that the exogenous target gene was successfully expressed in the wild rice callus, and the transient expression rate of GFP was calculated to be 82%.

### Test Example 1

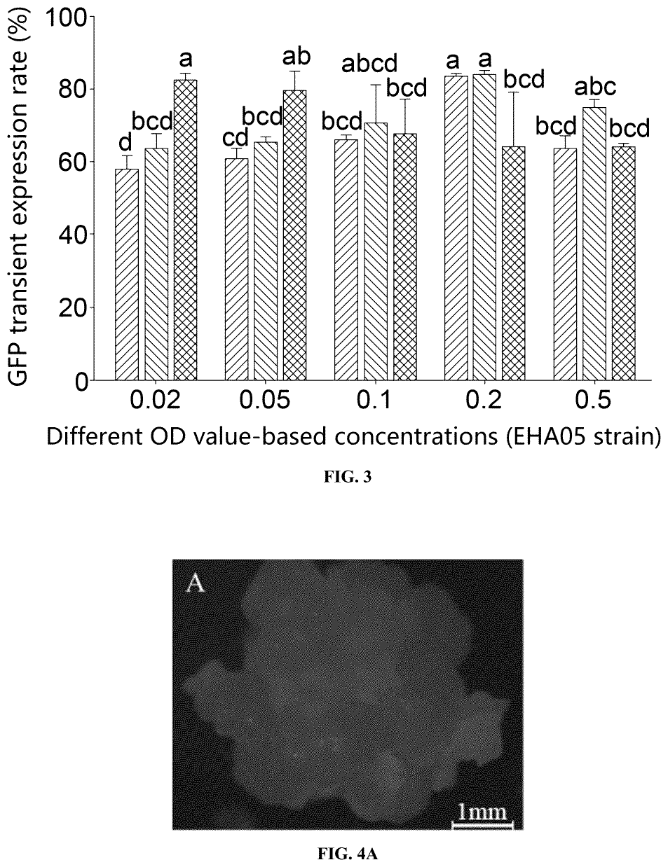

Test steps: EHA105-positive Agrobacterium bacterial solutions of different concentrations were prepared. The OD values of the bacterial solutions at a wavelength of 600 nm were: 0.02, 0.05, 0.1, 0.2, and 0.5. The callus separately infected with each concentration (OD value) of bacterial solution was co-cultured at three different co-culture times (2 d, 3 d, and 4 d). The results were shown in FIG. 3:

The effects of EHA105-positive Agrobacterium bacterial solution concentrations (OD values 0.02, 0.05, 0.1, 0.2, and 0.5) and different co-culture times (2 d, 3 d, and 4 d) on the transient expression rate of callus were compared. The results showed that different bacterial solution infection concentrations and different co-culture times showed significant differences. When being co-cultured for 2 d and 3 d, the transient expression rate of GFP showed an overall trend of increasing and then decreasing as the concentration of bacterial solution increased; after 4 d of co-culture, the overall transient expression rate of GFP showed a decreasing trend. After 3 d of co-culture, when the OD value of the bacterial solution at 600 nm wavelength was 0.2, the maximum transient expression rate of GFP was 84%; after 2 d of co-culture, when the OD value of the bacterial solution at 600 nm wavelength was 0.2, the maximum transient expression rate of GFP was 83%; after 4 d of co-culture, when the OD value of the bacterial solution at 600 nm wavelength was 0.02, the maximum transient expression rate of GFP was 82%.

When being co-cultured for 3 d to 4 d, the transient expression rate of GFP also changed as the concentration of bacterial solution continued to increase. However, the transient expression rate of GFP basically exceeded 60%, indicating that there was a relatively high transient expression rate.

### Test Example 2

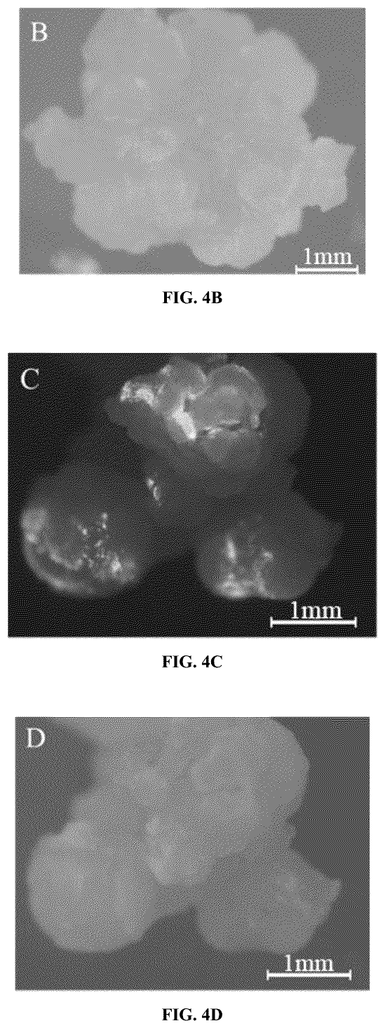

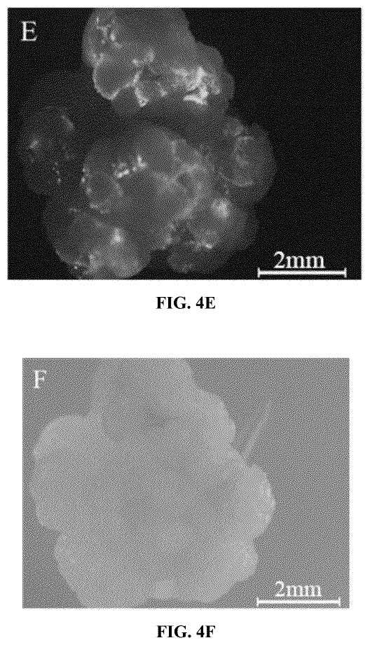

The recovered callus separately obtained after co-culture for 2 d, 3 d, and 4 d were placed under a stereo fluorescence microscope to observe the infection status, as shown in FIG. 4. By observing the callus after co-culture for 2 d (FIG. 4A and FIG. 4B), 3 d (FIG. 4C and FIG. 4D), and 4 d (FIG. 4E and FIG. 4F) under a stereo fluorescence microscope, it was found that there were significant differences in fluorescence expression level for different days of co-culture. The callus obtained after co-culture for 2 d had almost no GFP spots; while as the number of co-culture days increased, the number of GFP spots gradually increased after 3 d and 4 d of co-culture, and there was obvious green fluorescence.

The above description is only preferred examples of the present disclosure, and is not intended to limit the present disclosure. Although the present disclosure is expounded with reference to the above examples, a person skilled in the art can still make modifications on the technical solution described in the above examples or equivalent substitutions on some technical features of the technical solution. Any modification, equivalent substitution, improvement, etc. within the spirit and principles of the present disclosure shall fall within the scope of protection of the present disclosure.

What is claimed is:

1. A method for gene expression by transient transformation of a wild rice seed using Agrobacterium, comprising the following steps:
(1) inoculating a sterilized wild rice seed into an induction medium to allow induction culture of a callus, conducting subculture, and selecting a resulting callus with a dense structure to allow pre-culture to obtain a pre-cultured callus;
(2) transforming a 1305Ubi-Ubi-GFP-H plasmid carrying a green fluorescent protein (GFP) gene into an Agrobacterium strain EHA105 or LBA4404 to allow culture at 28° C. for 2 d, selecting a resulting single clone to allow PCR and electrophoresis detection to obtain a positive Agrobacterium strain; transferring the positive Agrobacterium strain into a yeast extract broth (YEB) liquid medium to allow culture, subjecting an obtained bacterial solution to centrifugation to collect a bacterial cell, resuspending the bacterial cell in an infection buffer until a resulting bacterial suspension has an optical density (OD) value of 0.02 to 0.5 at a wavelength of 600 nm to obtain an infection bacterial solution; and
(3) infecting the pre-cultured callus with the infection bacterial solution, inoculating a resulting infected callus into a co-culture medium to allow co-culture in the dark at 22° C.±2° C. for 2 d to 4 d; transferring a resulting co-cultured callus into a recovery medium to allow recovery culture in the dark at 28° C.±2° C. for 3 d; observing an infection status of a recovered callus under a stereo fluorescence microscope, wherein the GFP gene serving as an exogenous target gene is successfully expressed in the callus of the wild rice seed if the recovered callus appears green fluorescence under ultraviolet light; and calculating a transient expression rate.

2. The method according to claim 1, wherein the Agrobacterium strain is any one selected from the group consisting of Agrobacterium strains EHA105 and LBA4404, the co-culture is conducted for 3 d, and the bacterial suspension has an OD value of 0.2 at a wavelength of 600 nm.

3. The method according to claim 1, wherein the Agrobacterium strain is any one selected from the group consisting of Agrobacterium strains EHA105 and LBA4404, the co-culture is conducted for 2 d, and the bacterial suspension has an OD value of 0.2 at a wavelength of 600 nm.

4. The method according to claim 1, wherein a preparation process of the sterilized wild rice seed in step (1) comprises: immersing a wild rice seed in 75% ethanol for 30 s to 60 s and in 20% sodium hypochlorite for 20 min in sequence, washing a resulting immersed wild rice seed with sterile water 5 to 7 times until an obtained washing solution is clear, and then immersing a resulting washed wild rice seed in the sterile water overnight; and
an embryo of the sterilized wild rice seed is inoculated into the induction medium to allow germination, and the induction culture of the callus is conducted at 28° C.±2° C. for 4 to 5 weeks in the dark to obtain an embryogenic callus; an embryogenic callus with a light yellow appearance and a dense structure is selected to allow the subculture on the induction medium in the dark at 28° C.±2° C.; a resulting subcultured embryogenic callus with a dense structure is selected to allow the pre-culture in the dark at 28° C.±2° C. for 7 d to 9 d.

5. The method according to claim 1, wherein the induction medium in step (1) comprises: 4.43 g of Murashige & Skoog basal salt mixture (MS salt), 1 mL of 2 mg/mL 2,4-D, 0.5 g of casein hydrolysate, 30 g of sucrose, 0.1 g of inositol, and 3 g of phytagel per liter of the induction medium with a pH value of 5.7.

6. The method according to claim 1, wherein 50 mg/mL kanamycin and 50 mg/mL rifampicin are added into the YEB liquid medium, and the centrifugation is conducted at 1,000 rpm to 2,000 rpm for 5 min in step (2).

7. The method according to claim 1, wherein each liter of the infection buffer in step (2) comprises 4 g of trace element-containing CHU'S N6 BA SAL SALT MIX (N6) 1 mL of 200 μM/mL acetosyringone, 200 μL of 10 mg/mL 2,4-D, 1 g of casein hydrolysate, 30 g of sucrose, 0.1 g of inositol, and 10 g of glucose.

8. The method according to claim 1, wherein the pre-cultured callus is infected with the infection bacterial solution for 20 min, and the infected callus is co-cultured on the co-culture medium for 3 d in step (3).

9. The method according to claim 1, wherein the co-culture medium in step (3) comprises: 4 g of trace element-containing N6, 1 mL of 200 μM/mL acetosyringone, 200 μL of 10 mg/mL 2,4-D, 1 g of casein hydrolysate, 30 g of sucrose, 0.1 g of inositol, 10 g of glucose, and 3 g of phytagel per liter of the co-culture medium with a pH value of 5.7.

10. The method according to claim 1, wherein the recovery medium in step (3) comprises: 4.43 g of MS salt, 0.5 g of casein hydrolysate, 2 mL of 2 mg/mL 2,4-D, 100 μL of 1 mg/mL 6-BA, 2 mL of 200 mg/mL Timentin, 0.1 g of inositol, 30 g of sucrose, and 3 g of phytagel per liter of the recovery medium with a pH value of 5.7.
